(19) United States
(12) Patent Application Publication
(10) Pub. No.: US 2014/0221848 A1
(43) Pub. Date: Aug. 7, 2014

(54) BIOLOGICAL INFORMATION DETECTING DEVICE, BIOLOGICAL INFORMATION DETECTING METHOD, AND BIOLOGICAL INFORMATION DETECTION PROGRAM

(71) Applicant: CASIO COMPUTER CO., LTD., Tokyo (JP)

(72) Inventor: Tomoaki NAGASAKA, Tokyo (JP)

(73) Assignee: CASIO COMPUTER CO., LTD., Tokyo (JP)

(21) Appl. No.: 14/172,577

(22) Filed: Feb. 4, 2014

(30) Foreign Application Priority Data
Feb. 6, 2013 (JP) 2013-021247

(51) Int. Cl. A61B 5/024; A61B 5/11

(52) U.S. Cl. CPC A61B 5/02438; A61B 5/02416; A61B 5/1118
USPC 600/479; 600/500

(57) ABSTRACT

A biological information detecting device of the present invention, which detects the pulse of a user, includes a detecting section which outputs an observation signal detected based on a pulse wave of at least one observation site of the user and an acceleration measuring section which outputs a plurality of acceleration signals in each of a plurality of axial directions measured along with the user's movement. Based on a comparison between the observation signal and a composite acceleration signal obtained by combining the acceleration signals based on a plurality of parameters, the device estimates a specific value of each of the parameters corresponding to acceleration components of the observation signal based on the user's movement, and calculates the pulse rate of the user from a difference value obtained by subtracting a specific composite acceleration signal corresponding to the specific value of each of the estimated parameters from the observation signal.

### CROSS-REFERENCE TO RELATED APPLICATION

This application is based upon and claims the benefit of priority from the prior Japanese Patent Application No. 2013-021247, filed Feb. 6, 2013, the entire contents of which are incorporated herein by reference.

### BACKGROUND OF THE INVENTION

1. Field of the Invention

The present invention relates to a biological information detecting device, a biological information detecting method, and a biological information detection program. Specifically, the present invention relates to a biological information detecting device which has a pulse measuring function for measuring a pulse and is worn on a human body during exercise, a biological information detecting method, and a biological information detection program.

2. Description of the Related Art

In recent years, because of rising health consciousness, more and more people are performing daily exercises, such as running, walking, and cycling, to maintain their wellness or improve their health condition. These people measure and record various biological information in order to grasp their health conditions and exercise status.

This biological information for grasping the status of a human body includes various physiological indexes. As a physiological index, for example, a heart rate is well known, which indicates the number of heartbeats for one minute.

As a heart rate measuring method, an electrocardiogram method is generally known. In this electrocardiogram method, a plurality of electrodes have to be worn on the chest. However, by the electrodes being worn, activities in daily life or during exercise may be restricted, and wearing these electrodes is burdensome, which may place a heavy burden on measurement device users.

For this reason, a method is often used today in which, as a physiological index by which the measurement can be more easily performed, a pulse rate is used in place of a heart rate.

A known example of a pulse rate measuring method is photoplethysmography (or an optical pulse wave detection method). The principle of photoplethysmography is, in brief, that light absorption characteristics of hemoglobin in blood are used to detect an observation signal corresponding to a pulse wave. That is, the pulse wave represents pressure change in arteries occurred by heartbeats and propagated to peripheral arteries as undulation. By transmitting light such as infrared rays through the skin to irradiate blood in peripheral arteries and measuring temporal change in the intensity of reflected light from the transmitted light dispersed by blood as an observation signal, a pulse wave indicating undulatory change in the flow rate of blood in peripheral arteries can be detected. According to this photoplethysmography, a pulse wave can be obtained from a finger, earlobe, wrist, or the like and, based on the pulse wave, a pulse can be easily found.

However, blood flow changes by the movement of a body in daily life or during exercise. Therefore, there is a problem in photoplethysmography in that measurement is greatly influenced by blood flow change due to body movement (body movement noise), and this body movement noise is disadvantageously mixed into an observation signal.

By contrast, as a method for removing the signal components of body movement noise from an observation signal where a pulse wave signal and the body movement noise are mixed and acquiring the pulse wave signal, a method is described in Japanese Patent Application Laid-Open (Kokai) Publication No. 2003-102694 in which body movement noise is regarded as an acceleration signal obtained by an accelerometer and a differential signal between the observation signal and the acceleration signal is taken as a pulse wave signal.

In this pulse wave signal obtaining method, since signal processing is performed by an acceleration signal regarded as equivalent to body movement noise, a pulse wave signal can be obtained by relatively simple signal processing.

However, according to verifications by the inventor of the present invention, an acceleration signal obtained when a human body is moving and body movement noise are not necessarily identical. For example, it has been found that the acceleration signal and the body movement noise have different amplitudes and there is a time difference (time lag) between the time when the acceleration occurs and the time when its influence appears on an observation signal. Moreover, it has been found that this time difference is not constant and varies according to change in the movement status of the human body and change in the pulse measurement position.

In the above-described method, these points are not considered at all. As a result, in the above-described method, noise components due to body movement included in an observation signal are not appropriately removed, and a pulse rate when a human body is moving cannot be accurately measured.

### SUMMARY OF THE INVENTION

The present invention can advantageously provide a biological information detecting device, a biological information detecting method, and a biological information detection program capable of detecting a precise pulse wave by appropriately estimating a body-movement noise component occurred along with the movement of a user which is in an observation signal detected based on the pulse wave of the user.

In accordance with one aspect of the present invention, there is provided a biological information detecting device comprising: a detecting section which outputs an observation signal detected based on a pulse wave of at least one observation site of a user; an acceleration measuring section which outputs a plurality of acceleration signals which are corresponding to a plurality of different axial directions and are measured along with a movement of the user; a parameter estimating section which estimates, based on a comparison between the observation signal and a composite acceleration signal obtained by combining the plurality of acceleration signals based on a plurality of parameters, a specific value of each of the parameters corresponding to acceleration components obtained in response to the movement of the user; and a pulse rate calculating section which calculates a pulse rate of the user as a biological information from a difference value obtained by subtracting a specific composite acceleration signal corresponding to the specific value of each of the parameters from the observation signal.

In accordance with another aspect of the present invention, there is provided a biological information detecting method comprising: a step of obtaining an observation signal based on a pulse wave of at least one observation site of a user, and obtaining a plurality of acceleration signals which are corresponding to a plurality of different axial directions along with a movement of the user; a step of estimating, based on a comparison between the observation signal and a composite acceleration signal obtained by combining the plurality of acceleration signals based on a plurality of parameters, a specific value of each of the parameters corresponding to acceleration components obtained in response to the movement of the user included in the observation signal; and a step of calculating a pulse rate of the user as a biological information from a difference value obtained by subtracting a specific composite acceleration signal corresponding to the specific value of each of the parameters from the observation signal.

In accordance with another aspect of the present invention, there is provided a non-transitory computer-readable storage medium having stored thereon a biological information detection program that is executable by a computer, the program being executable by the computer to perform functions comprising: processing for obtaining an observation signal based on a pulse wave of at least one observation site of a user, and obtaining a plurality of acceleration signals which are corresponding to a plurality of different axial directions set in advance along with a movement of the user; processing for estimating, based on a comparison between the observation signal and a composite acceleration signal obtained by combining the plurality of acceleration signals based on a plurality of parameters, a specific value of each of the parameters corresponding to acceleration components obtained in response to the movement of the user included in the observation signal; and processing for calculating a pulse rate of the user as a biological information from a difference value obtained by subtracting a specific composite acceleration signal corresponding to the specific value of each of the parameters from the observation signal.

The above and further objects and novel features of the present invention will more fully appear from the following detailed description when the same is read in conjunction with the accompanying drawings. It is to be expressly understood, however, that the drawings are for the purpose of illustration only and are not intended as a definition of the limits of the invention.

### BRIEF DESCRIPTION OF THE DRAWINGS

FIG. 1A and FIG. 1B are schematic diagrams depicting an example in which a biological information detecting device according to the present invention has been worn and a structural example of the outer appearance thereof;

FIG. 2A, FIG. 2B and FIG. 2C are schematic diagrams depicting structural examples of the measurement surface of the biological information detecting device according to the present invention;

FIG. 3 is a block diagram depicting a structural example of a biological information detecting device according to a first embodiment;

FIG. 4 is a flowchart of a standing still pulse wave measuring operation that is performed in a biological information detecting method of the biological information detecting device according to the first embodiment;

FIG. 5A, FIG. 5B, FIG. 5C and FIG. 5D are conceptual diagrams showing an example of multipoint pulse wave observation in the standing still pulse wave measuring operation according to the first embodiment;

FIG. 6 is a waveform diagram showing an example of a pulse wave signal obtained by the standing still pulse wave measuring operation according to the first embodiment;

FIG. 7 is a flowchart of a movement pulse wave measuring operation that is performed in the biological information detecting method of the biological information detecting device according to the first embodiment;

FIG. 8 is a conceptual diagram for describing an extreme value interval calculated in the movement pulse wave measuring operation according to the first embodiment;

FIG. 9A, FIG. 9B and FIG. 9C are waveform diagrams showing an example of each signal obtained by the movement pulse wave measuring operation according to the first embodiment;

FIG. 10 is a flowchart of time lag and rotation angle estimation processing that is performed in the movement pulse wave measuring operation according to the first embodiment;

FIG. 11 is a conceptual diagram for describing three axial directions defined in the movement pulse wave measuring operation according to the first embodiment;

FIG. 12 is a diagram depicting an example of a normalized cross-correlation coefficient calculated by the time lag and rotation angle estimation processing according to the first embodiment;

FIG. 13 is a diagram depicting an example of the transition of a rotation angle and a maximum value obtained by the time lag and rotation angle estimation processing according to the first embodiment;

FIG. 14 is a flowchart of amplitude estimation processing that is performed in the movement pulse wave measuring operation according to the first embodiment;

FIG. 15A and FIG. 15B are schematic diagrams depicting a structural example of the measurement surface of a biological information detecting device according to a second embodiment; and

FIG. 16 is a flowchart of a movement pulse wave measuring operation that is performed in a biological information detecting method according to the second embodiment.

### DETAILED DESCRIPTION OF THE PREFERRED EMBODIMENTS

Embodiments of a biological information detecting device, a biological information detecting method, and a biological information detection program according to the present invention will hereinafter be described in detail with reference to the drawings.

### First Embodiment

### Biological Information Detecting Device

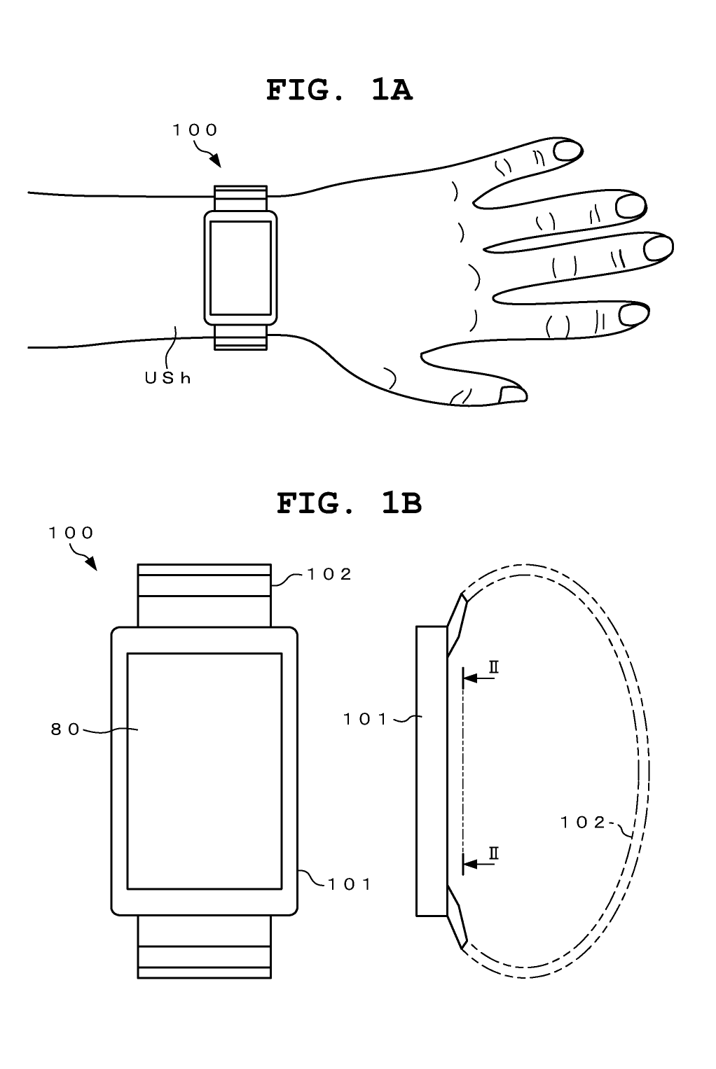

FIG. 1A and FIG. 1B are schematic diagrams depicting an example in which a biological information detecting device according to the present invention has been worn and a structural example of the outer appearance thereof.

Here, FIG. 1A is a schematic diagram depicting a state in which the biological information detecting device according to the present invention has been worn on the human body, and FIG. 1B is a schematic structural diagram depicting the front surface and a side surface of the biological information detecting device according to the present invention.

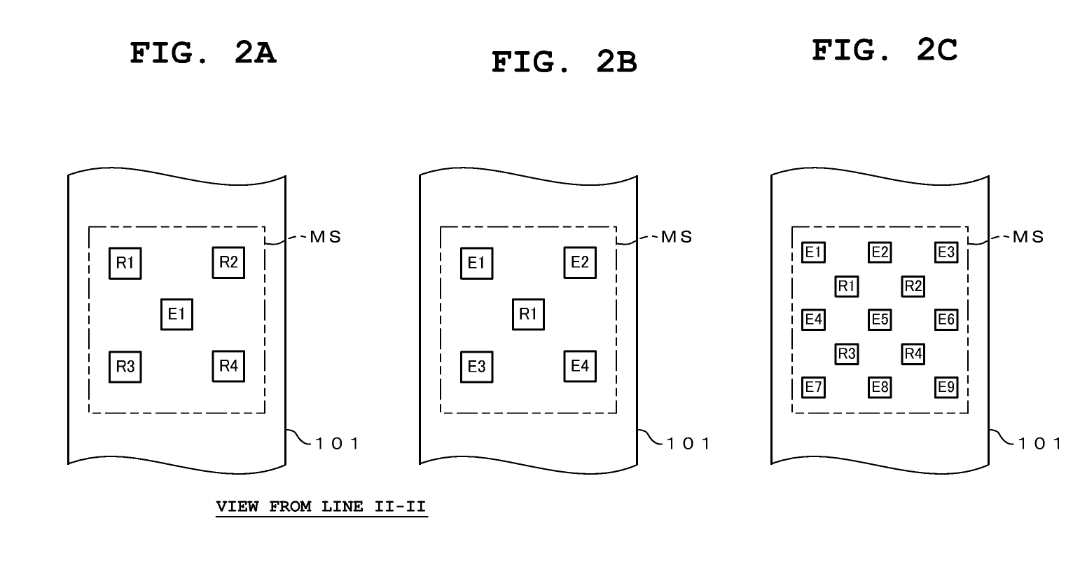

FIG. 2A, FIG. 2B and FIG. 2C are schematic diagrams depicting structural examples of a measurement area of the biological information detecting device according to the present invention.

The biological information detecting device 100 according to the present invention has an outer appearance of, for example, a wristwatch (or a wristband) that is worn on a wrist or the like of a user US, as depicted in FIG. 1A.

This biological information detecting device 100 mainly includes, for example, a device body 101 having functions for measuring a pulse of the user US and providing predetermined information to the user US, and a belt section 102 that is wound around a wrist USh of the user US so that the device body 101 is worn on and comes in close contact with the wrist USh, as depicted in FIG. 1B.

In a predetermined area of a surface of the device body 101 which comes in contact with the wrist USh (the right side surface indicated by line II-II in FIG. 18), a measurement area MS is provided.

In this measurement area MS, for example, one or plurality of light-emitting elements E1 to E9 and one or plurality of light-receiving elements R1 to R4 are two-dimensionally arranged in a predetermined pattern, as depicted in FIG. 2A to FIG. 2C.

For example, the light-emitting elements and the light-receiving elements are arranged in the measurement area MS as depicted in FIG. 2A to FIG. 2C.

In the arrangement depicted in FIG. 2A, a plurality of (four) light-receiving elements R1 to R4 have been arranged to surround one light-emitting element E1. That is, the light-emitting element and the light-receiving elements have been arranged such that they have a one-to-many relation.

In the arrangement depicted in FIG. 2B, a plurality of (four) light-emitting elements E1 to E4 have been arranged to surround one light-receiving element R1. That is, the light-emitting elements and the light-receiving element have been arranged such that they have a many-to-one relation.

In the arrangement depicted in FIG. 2C, a plurality of light-emitting elements E1 to E9 have been arranged to surround each of a plurality of (four) light-receiving elements R1 to R4. That is, the light-emitting elements and the light-receiving elements have been arranged such that they have a many-to-many relation.

As such, in the structure of the present embodiment, one or plurality of light-emitting elements and one or plurality of light-receiving elements are arranged such that the number of light-emitting elements or light-receiving elements is not one.

Note that the number and arrangement of light-emitting elements and light-receiving elements that are arranged in the measurement area MS are not limited to those of the patterns in FIG. 2A to FIG. 2C, and any number of light-emitting elements and light-receiving elements may be arranged in an arbitrary pattern such as a staggered shape, lattice shape, or arc shape.

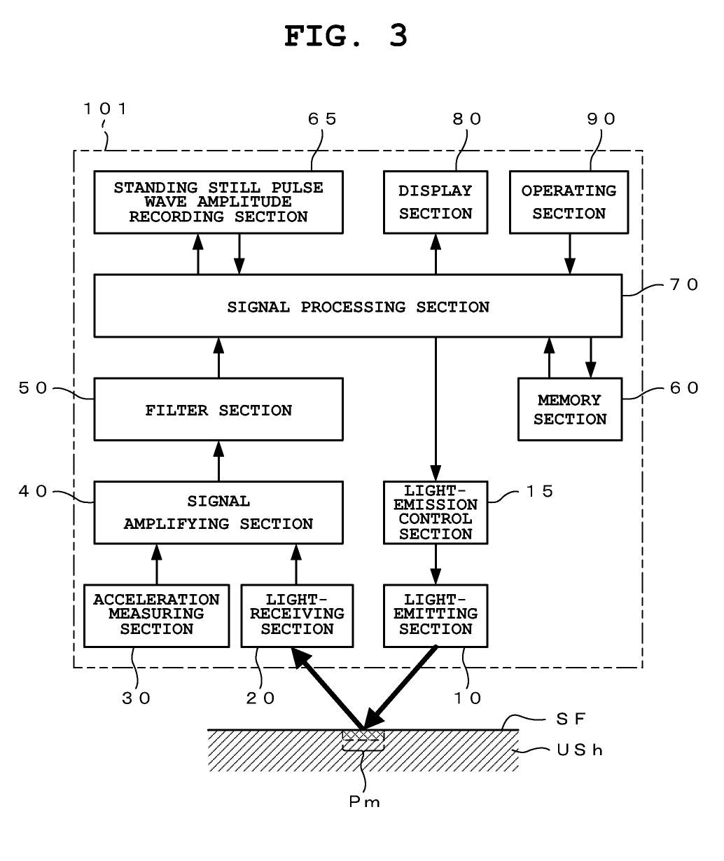

FIG. 3 is a block diagram depicting a structural example of the biological information detecting device according to the present embodiment.

Specifically, the biological information detecting device 100 mainly includes, for example, a light-emitting section (a detecting section) 10, a light-emission control section 15, a light-receiving section (a detecting section) 20, an acceleration measuring section 30, a signal amplifying section 40, a filter section 50, a memory section 60, a standing still pulse wave amplitude recording section (a storage section) 65, a signal processing section (an observation signal selecting section, a parameter estimating section, and a pulse rate calculating section) 70, a display section 80, and an operating section 90, as depicted in FIG. 3.

The light-emitting section 10 has one or plurality of light-emitting elements E1 to E9 described above, which are arranged in a predetermined pattern in the measurement area MS on the surface of the device body 101 which comes in contact with the wrist USh, as depicted in FIG. 2A to FIG. 2C.

As the light-emitting elements E1 to E9, for example, light-emitting diodes (LED) or the like can be applied. These light-emitting elements E1 to E9 emit visible light with a predetermined light-emission intensity (or a light-emission amount) in accordance with drive control by the light-emission control section 15 described below, and irradiate the skin surface (body surface) SF of the wrist USh with the emitted light.

Here, since the transmittance of visible light into the body is low, a reflective-type pulse-wave detecting method using visible light is resistant to the influence of reflected light from blood flows of veins and arteries present in deep parts inside the body and is resistant to the influence of propagation time lag of pulsation occurring in blood vessels due to the blood flow length.

Note that, as visible light emitted from the light-emitting elements, green visible light having a wavelength of about 525 nm can be favorably applied.

The light-emission control section 15 causes one or plurality of light-emitting elements E1 to E9 constituting the light-emitting section 10 to individually emit light in a predetermined lighting pattern (that is, in a predetermined sequence and with a predetermined light-emission intensity) in accordance with control by the signal processing section 70 described below.

The light-receiving section 20 has one or plurality of light-receiving elements R1 to R4 described above, which are arranged in a predetermined pattern in the measurement area MS of the device body 101, as depicted in FIG. 2A to FIG. 2C.

As the light-receiving elements R1 to R4, for example, phototransistors or illuminance sensors can be applied. The light-receiving elements R1 to R4 receive lights which have been individually emitted from one or plurality of light-emitting elements E1 to E9, applied to an observation site Pm where a pulse wave is observed, and dispersed by blood in a blood vessel near the observation site Pm as reflected lights, and thereby outputs an output signal (an observation signal) according to the light-receiving amount.

The acceleration measuring section 30 has a triaxial acceleration sensor. This triaxial acceleration sensor outputs, as an acceleration signal, a ratio of change in the moving speed (acceleration) of the biological information detecting device 100 when the user US is moving.

The acceleration signal outputted from the acceleration measuring section 30 is formed of three acceleration signals corresponding to three axial directions formed of X axis, y axis, and z axis and orthogonal to each other, which will be described further below.

The signal amplifying section 40 amplifies an observation signal obtained by the light-receiving section 20 and an acceleration signal measured by the acceleration measuring section 30 to a predetermined signal level suitable for signal processing in the signal processing section 70 described below.

Among the observation signal and the acceleration signal amplified by the signal amplifying section 40, signal components of a predetermined frequency band pass through the filter section 50 and supplied to the signal processing section 70.

The memory section 60 has, for example, a memory for data storage (hereinafter referred to as a “data memory”), a memory for program storage (hereinafter referred to as a “program memory”), and a memory for work data storage (hereinafter referred to as a “work memory”).

The data memory has a non-volatile memory such as a flash memory, and stores (records) an observation signal obtained by the light-receiving section 20 and an acceleration signal measured by the acceleration measuring section 30 when the user US is moving or exercising in a predetermined storage area in association with time data.

The program memory includes a ROM (Read Only Memory), and has stored therein a control program for achieving a predetermined function in each section (such as the light-emitting section 10, the light-receiving section 20, the acceleration measuring section 30, and the display section 80 and the operating section 90 described below) of the biological information detecting device 100 and an algorithm program for achieving a function for calculating a pulse rate based on the above-described observation signal and acceleration signal.

The work memory has a RAM (Random Access Memory), and temporarily stores various data that is used or generated when the control program or the algorithm program is executed.

Note that the entire or part of the data memory may be in a form of a removable storage medium such as a memory card, and may be structured to be removable from the device body 101 of the biological information detecting device 100.

The standing still pulse wave amplitude recording section 65 stores (records) the amplitude of the signal wave (pulse wave) of an observation signal obtained by the light-receiving section 20 when the user US is not moving, such as when he or she is standing still or at rest, in a predetermined storage area in association with time data.

The signal processing section 70, which is a CPU (Central Processing Unit) or a MPU (Microprocessor Unit), performs processing in accordance with the control program stored in the memory section 60, and thereby controls an operation of storing and reading various data in the memory section 60, an operation of displaying various information in the display section 80, an operation of detecting an input operation in the operating section 90, and the like.

The signal processing section 70 performs processing in accordance with the algorithm program stored in the memory section 60, and thereby performs, for example, an operation of calculating a pulse rate based on an observation signal obtained by the light-receiving section 20 and an acceleration signal measured by the acceleration measuring section 30, as will be explained in the description of a biological information detecting method.

Note that the control program and the algorithm program which are executed in the signal processing section 70 may be incorporated in advance in the signal processing section 70.

The display section 80 has, for example, a display device such as a liquid-crystal display panel or an organic EL display panel capable of color or monochrome display, and displays at least a pulse rate calculated by the signal processing section 70.

Note that the display section 80 may display, in addition to or in place of a pulse rate, a pulse wave (pulse wave form data), moving speed, footstep count, current time, and the like by using characters, numerical information, image information, and the like.

Here, for example, the pulse wave form data contains various information regarding blood flows.

That is, a configuration may be adopted in which pulse wave data is applied as important parameters for judging, for example, health and physical condition (such as judgment regarding clots in blood vessels, vascular age, and tension status), exercise status, and the like, and the judgment results are displayed on the display section 80 with a specific character, numerical information, image information, light-emission pattern, and the like.

In the present embodiment, only the display section 80 is described as an output interface for providing or notifying the user US with various information. However, the present embodiment is not limited thereto. For example, in addition to the display section 80, another interface may be provided to the present embodiment, such as an acoustic section including a buzzer or loudspeaker which generates a specific sound or audio message or a vibrating section which vibrates with a specific vibration pattern.

The operating section 90 includes a button switch, a slide switch, a keyboard, a touch panel arranged or integrally formed on the front surface of the display section 80, and the like. This operating section 90 is used to select or perform various operations, such as a power supply ON/OFF operation in the biological information detecting device 100, an operation of measuring a pulse wave and acceleration, and a display operation in the display section 80, and to input set values and the like.

### (Biological Information Detecting Method)

Next, the biological information detecting method in the above-described biological information detecting device is described.

In the biological information detecting method in the above-structured biological information detecting device, a standing still pulse wave measuring operation for obtaining a pulse wave observation signal when the user is not moving and a movement pulse wave measuring operation of calculating a pulse rate based on a pulse wave observation signal and an acceleration signal obtained while the user is moving are mainly performed.

### (Standing Still Pulse Wave Measuring Operation)

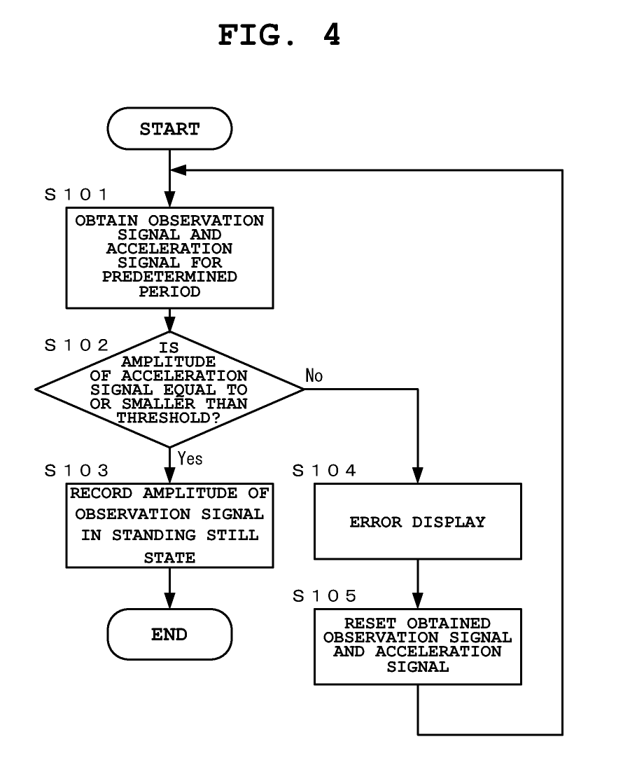

FIG. 4 is a flowchart of a standing still pulse wave measuring operation that is performed in the biological information detecting method of the biological information detecting device according to the present embodiment.

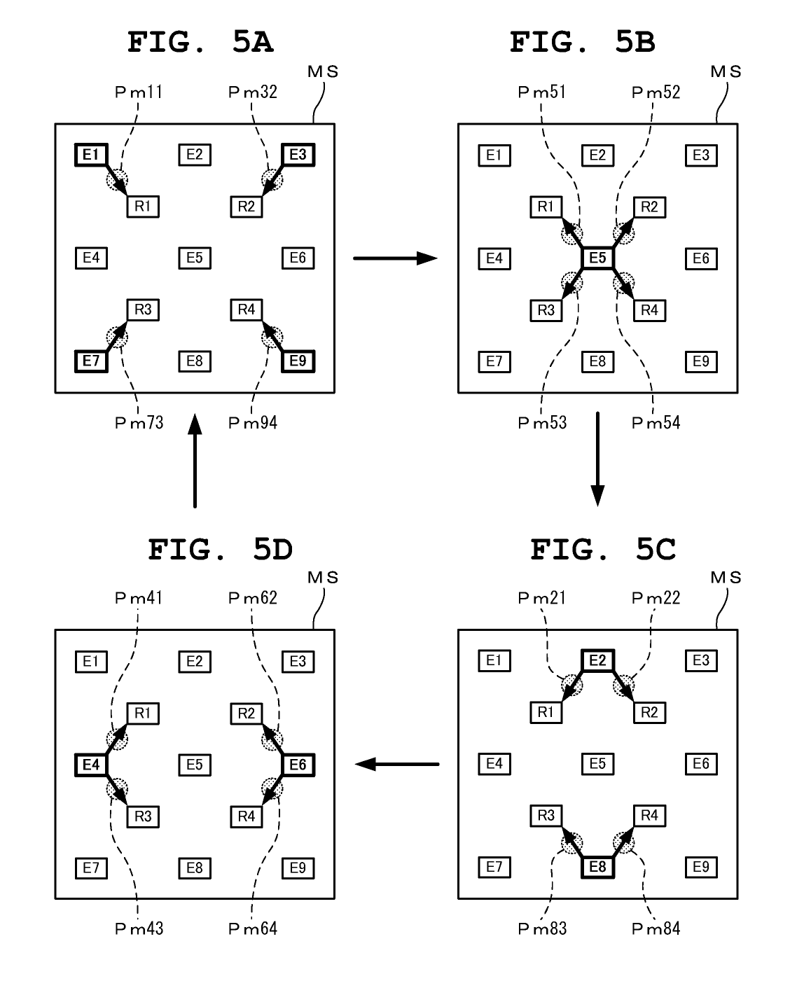

FIG. 5A, FIG. 5B, FIG. 5C and FIG. 5D are conceptual diagrams showing an example of multipoint pulse wave observation in the standing still pulse wave measuring operation according to the present embodiment.

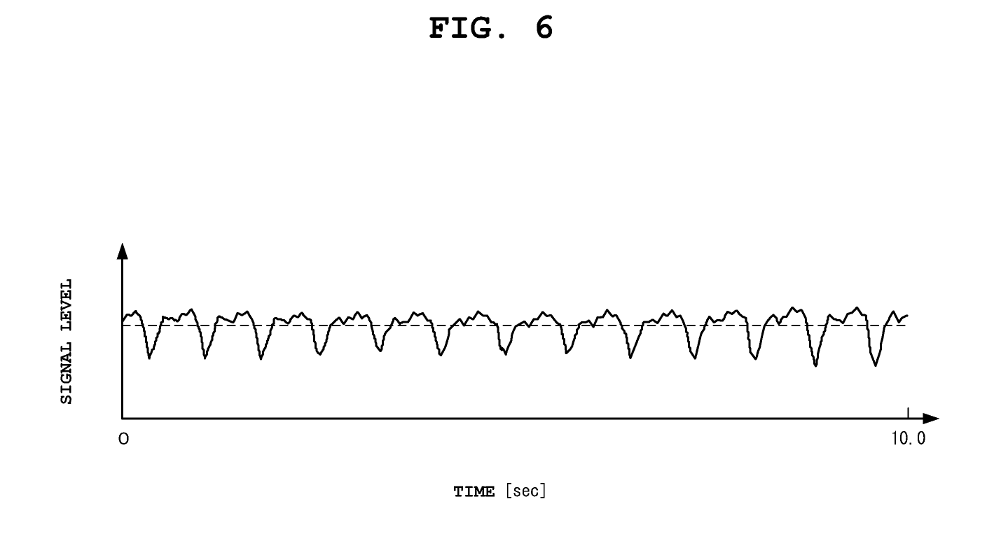

FIG. 6 is a waveform diagram showing an example of a pulse wave signal obtained by the standing still pulse wave measuring operation according to the present embodiment.

In the standing still pulse wave measuring operation, as depicted in FIG. 4, the signal processing section 70 first obtains a pulse wave observation signal and an acceleration signal when the user US is in a standing still or resting state and not making a movement such as an exercise, for a predetermined period (Step S101).

Specifically, the signal processing section 70 causes the display section 80 to display character information, image information, or the like indicating that a pulse wave in the standing still state is to be measured, and thereby prompts the user US to keep a standing still or resting state.

Next, the signal processing section 70 specifies a combination of a specific light-emitting element of the light-emitting section 10 and a specific light-receiving element of the light-receiving section 20, and causes the light-emission control section 15 to control the specified light-emitting element to emit light with a predetermined light-emission intensity. As a result, an area on the skin surface SF of the user US where a pulse wave is to be observed (the observation site Pm) is irradiated with the light emitted from the light-emitting element.

Part of the irradiated light is dispersed by blood in blood vessels near the observation site Pm, and emitted from the skin surface SF as reflected light.

This reflected light is received by the specified light-receiving element. Then, an output signal according to the light-receiving amount of the light-receiving element is outputted as an observation signal to the signal processing section 70 via the signal amplifying section 40 and the filter section 50.

Here, an example of the operation of obtaining an observation signal with a combination of a specific light-emitting element and a specific light-receiving element is described in detail by taking an example in which the arrangement of the light-emitting elements and the light-receiving elements has the arrangement pattern of the light-emitting elements E1 to E9 and the light-receiving elements R1 to R4 depicted in FIG. 2C.

First, the signal processing section 70 specifies, for example, a combination of the light-emitting element E1 and the light-receiving element R1, a combination of the light-emitting element E3 and the light-receiving element R2, a combination of the light-emitting element E7 and the light-receiving element R3, and a combination of the light-emitting element E9 and the light-receiving element R4, as depicted in FIG. 5A.

Next, the signal processing section 70 causes the light-emission control section 15 to control the light-emitting elements E1, E3, E7 and E9 to emit light with a predetermined light-emission intensity so as to irradiate observation sites Pm11, Pm32, Pm73 and Pm94 on the skin surface SF with light, and its reflected light is received by each of the light-receiving elements R1, R2, R3 and R4.

As a result, the signal processing section 70 obtains pulse wave observation signals of the observation sites Pm11, Pm32, Pm73 and Pm94 on the skin surface SF in the standing still state.

Here, the operations of obtaining observation signals of the observation sites Pm11, Pm32. Pm73 and Pm94 are sequentially performed in the order of, for example, the observation sites Pm11, Pm32, Pm73 and Pm94. Note that the observation signal obtaining operations for the observation sites Pm11, Pm32, Pm73 and Pm94 may be performed concurrently.

Next, the signal processing section 70 specifies, for example, a combination of the light-emitting element E5 and each of the light-receiving elements R1 to R4, as depicted in FIG. 5B.

Next, the signal processing section 70 causes the light-emission control section 15 to control the light-emitting element E5 to emit light with a predetermined light-emission intensity so as to irradiate each of observation sites Pm51, Pm52, Pm53 and Pm54 of the skin surface SF with light, and its reflected light is received by each of the light-receiving elements R1 to R4.

As a result, the signal processing section 70 obtains pulse wave observation signals of the observation sites Pm51 to Pm54 on the skin surface SF in the standing still state.

Here, the operations of obtaining observation signals of the observation sites Pm51 to Pm54 are sequentially performed in the order of the observation sites Pm51 to Pm54, as in the case of FIG. 5. Note that these operations for the observation sites Pm51 to Pm 54 may be performed concurrently.

Thereafter, similarly, the signal processing section 70 specifies a combination of the light-emitting element E2 and each of the light-receiving elements R1 and R2, a combination of the light-emitting element E8 and each of the light-receiving elements R3 and R4, a combination of the light-emitting element E4 and each of the light-receiving elements R1 and R3, and a combination of the light-emitting element E6 and each of the light-receiving elements R2 and R4, and obtains a pulse wave observation signal of each of observation sites Pm21, Pm22, Pm83, Pm84, and Pm41, Pm62, Pm43, and Pm64 in the standing still state, as depicted in FIG. 5C and FIG. 5D.

By this series of operations (multipoint observation), a pulse wave observation signal of each observation site between a light-emitting element and a light-receiving element adjacent to each other and arranged in the measurement area MS is acquired.

Also, the operation of obtaining a pulse wave observation signal is continuously performed for a period of time in which several to ten-odd waveforms indicating pulse waves are included, such as several seconds to several ten seconds.

On the other hand, concurrently with the operation of obtaining a pulse wave observation signal, the signal processing section 70 controls the acceleration measuring section 30 to measure the acceleration of the user US in three axial directions.

This operation of measuring triaxial acceleration is performed continuously while the operation of obtaining a pulse wave observation signal is being performed.

The triaxial acceleration measured by the acceleration measuring section 30 is outputted as an acceleration signal to the signal processing section 70 via the signal amplifying section 40 and the filter section 50.

The obtained pulse wave observation signals and acceleration signals are associated with each other based on time data, and stored in a predetermined area of the memory section 60.

Next, the signal processing section 70 judges whether the amplitude of each of the acceleration signals in three axial directions obtained at Step S101 is equal to or smaller than a predetermined threshold (Step S102).

Specifically, the signal processing section 70 reads out the acceleration signals obtained during the operation of obtaining a pulse wave observation signal from the memory section 60, and makes a judgment regarding each of the acceleration signals in three axial directions whether a maximum amplitude value (maximum amplitude) indicating a difference value between a maximum value and a minimum value of a signal waveform is equal to or smaller than a predetermined threshold value relevant to a standing still or resting state where the user US is not making a movement such as an exercise. That is, by using this threshold value, the signal processing section 70 judges whether the user US is in a standing still or resting state or in a moving state.

Here, the threshold value for judging whether the user US is in a standing still or resting state or in a moving state can be set at, for example, approximately 5% of amplitude at the time of running or the like.

This threshold value may be set based on, for example, a previous acceleration signal obtained while the user US is moving, or an acceleration signal of a general motion obtained from a large indefinite number of samples. Also, an arbitrary value may be set therefor while the user US is in a standing still or resting state.

As will be described below, the inventor of the present application has found that acceleration signals in the z-axis direction hardly have an influence on pulse wave signals. For this reason, in the above-described judgment regarding whether the amplitude of each acceleration signal is equal to or smaller than the predetermined threshold, a judgment regarding the amplitude of an acceleration signal in the x-axis direction is not required to be made.

When judged at Step S102 that the amplitude of each of the obtained acceleration signals in the three axial directions is equal to or smaller than the threshold value, the signal processing section 70 judges that the user US is standing still or at rest, and causes the average value of the amplitudes of the pulse wave observation signals of the respective observation sites at that time to be recorded as the amplitude of the observation signal in the standing still state (Step S103).

Specifically, when judged that the amplitude of each of the obtained acceleration signals in three axial directions is equal to or smaller than the threshold value, the signal processing section 70 reads out a pulse wave observation signal of each observation site stored in the memory section 60 in association with the acceleration signal based on the time data, and calculates an average amplitude value, which is a difference value between the maximum value and the minimum value of these signal waveforms.

Then, the signal processing section 70 stores (records) the calculated average value in the standing still pulse wave amplitude recording section 65 as the amplitude of the observation signal in the standing still state, and ends the standing still pulse wave measuring operation.

On the other hand, when judged at Step S102 that the amplitude of each of the obtained acceleration signals in the three axial directions is larger than the threshold value, the signal processing section 70 judges that the user US is not standing still or at rest, and performs error display so as to prompt the user US to stand still (Step S104).

Specifically, when judged that one of the amplitudes of the obtained acceleration signals in three axial directions is larger than the threshold, the signal processing section 70 judges that the user US is not standing still or at rest, causes the display section 80 to display character information, image information, or the like for prompting the user US to stop his or her movement, such as exercise, and to stand still, and thereby prompts the user US to keep a standing still or resting state.

Next, the signal processing section 70 performs a reset operation of deleting or discarding the pulse wave observation signal and the acceleration signals stored in the memory section 60 (Step S105).

Then, after returning to Step S101, the signal processing section 70 again performs the series of processing (Steps S101 to S105) described above.

The signal waveform of an observation signal in a standing still state obtained by the above-described standing still pulse wave measuring operation, that is, a signal waveform formed of the average value of the amplitudes of pulse wave observation signals of the respective observation sites can be defined as substantially not containing body-movement noise due to the movement of the user US or being a pulse wave signal in a state where body-movement noise is substantially negligible. This pulse-wave signal has a waveform such as that depicted in FIG. 6.

FIG. 6 depicts an example of a signal waveform when a pulse wave in a standing still state is observed for ten seconds.

In FIG. 6, the vertical axis represents digital values obtained by A/D conversion of an observation signal obtained by the light-receiving section 20 (light-receiving elements).

### (Movement Pulse Wave Measuring Operation)

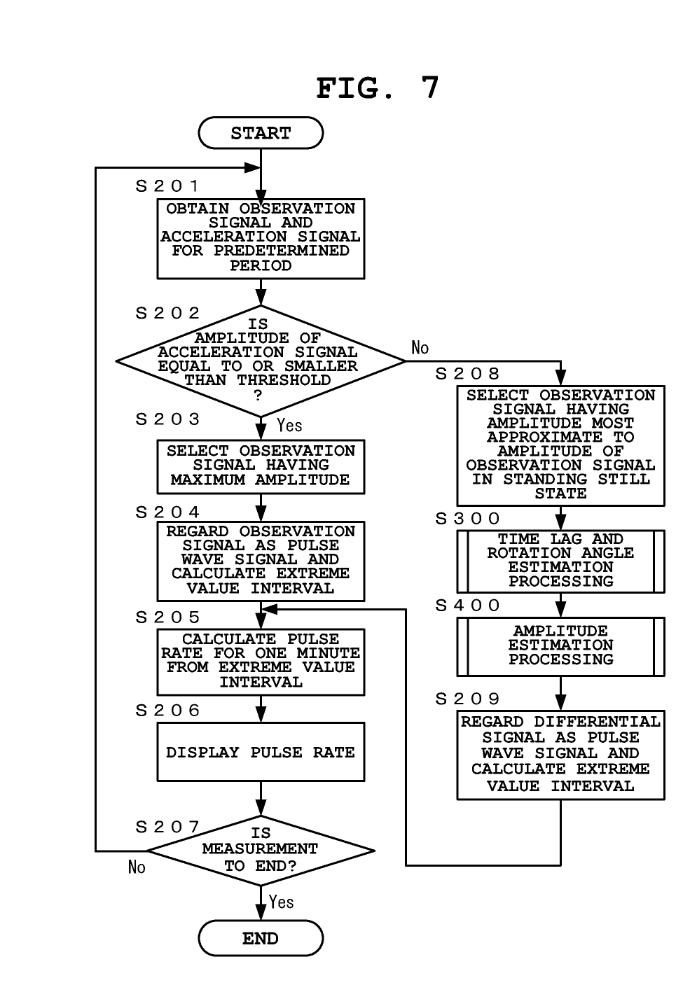

FIG. 7 is a flowchart of a movement pulse wave measuring operation that is performed in the biological information detecting method of the biological information detecting device according to the present embodiment.

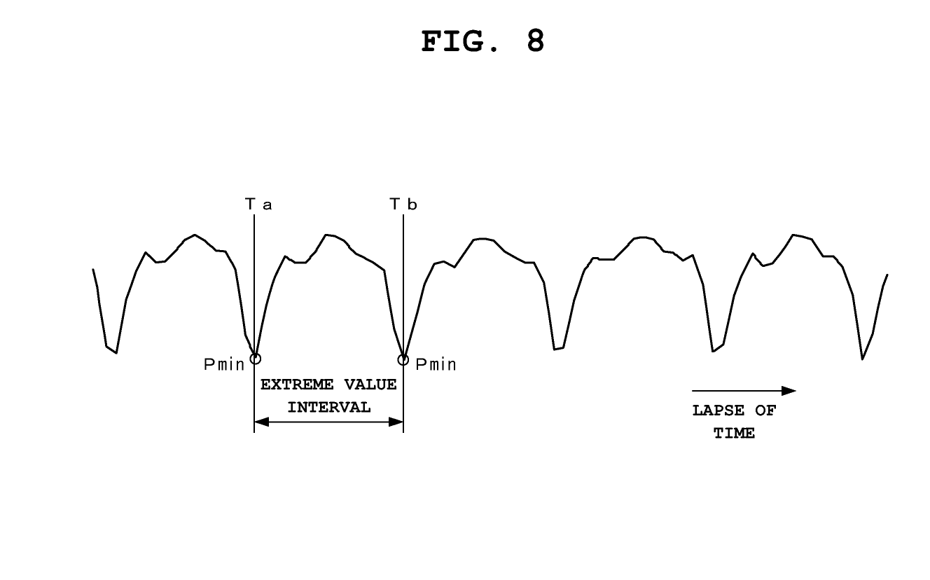

FIG. 8 is a conceptual diagram for describing an extreme value interval calculated in the movement pulse wave measuring operation according to the present embodiment.

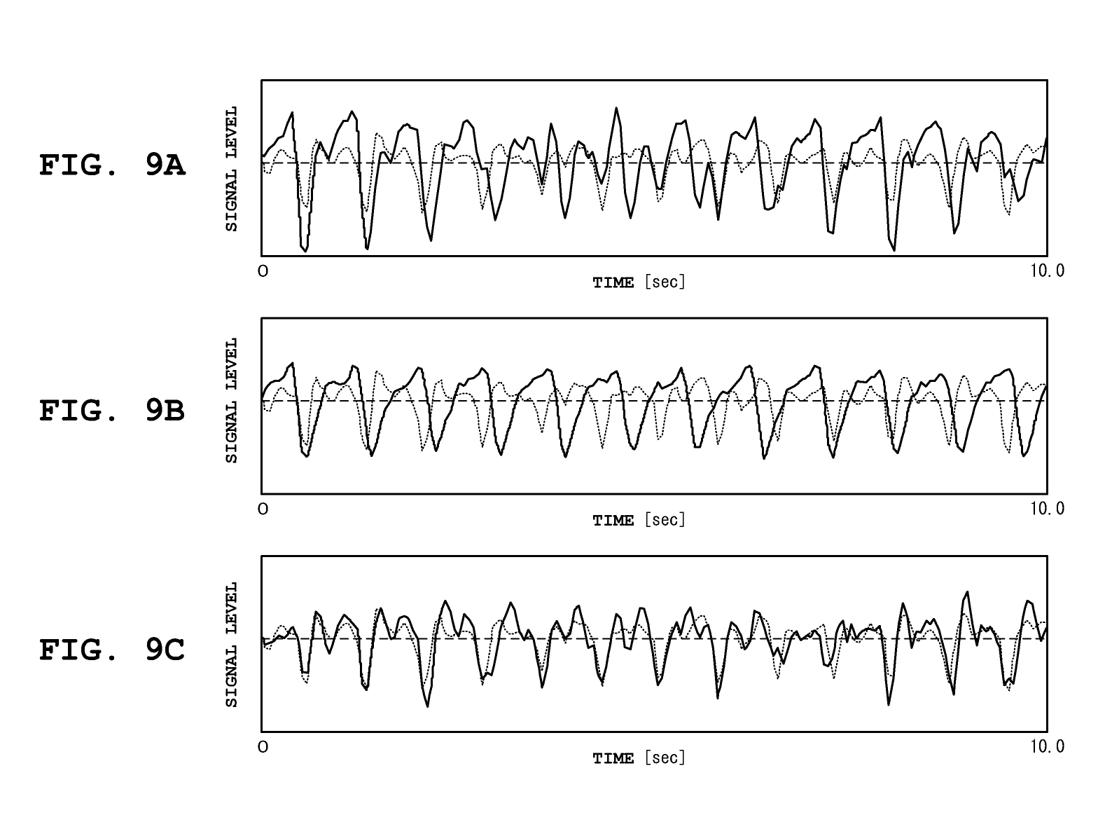

FIG. 9A, FIG. 9B and FIG. 9C are waveform diagrams showing an example of each signal obtained by the movement pulse wave measuring operation according to the present embodiment.

In the movement pulse wave measuring operation, as depicted in FIG. 7, the signal processing section 70 first obtains a pulse wave observation signal and an acceleration signal in a moving state where the user US is making a movement such as an exercise, for a predetermined period (Step S201).

Specifically, when the user US is making a movement such as an exercise, the signal processing section 70 obtains, for a predetermined period, a pulse wave observation signal of each observation site between a light-emitting element and a light-receiving element adjacent to each other and arranged in the measurement area MS, as in the case of the standing still pulse wave measuring operation.

Here, as with Step S101 of the above-described standing still pulse wave measuring operation, the operation of obtaining a pulse wave observation signal may be any operation as long as it is performed for an arbitrary period in which several to ten-odd waveforms indicating pulse waves are included. This period may be set the same as that of the standing still pulse wave measuring operation (for example, several seconds to several ten seconds), or may be set to a different period.

On the other hand, while the operation of obtaining a pulse wave observation signal is being performed, the signal processing section 70 continuously obtains acceleration signals in three axial directions caused by the movement of the user US.

The obtained pulse wave observation signals and acceleration signals are associated with each other based on time data, and stored in a predetermined area of the memory section 60.

Next, as with the above-described standing still pulse wave measuring operation, the signal processing section 70 judges whether the maximum value of the amplitudes (the maximum amplitude) of each of the acceleration signals in three axial directions obtained at Step S201 is equal to or smaller than a predetermined threshold value relevant to a standing still state (Step S202).

Next, when judged that the amplitude of the acceleration signal is equal to or smaller than the threshold value, the signal processing section 70 judges that the user US is standing still. Then, the signal processing section 70 regards (judges) a pulse wave signal having the maximum amplitude among pulse wave observation signals of the respective observation sites obtained at Step S201 as a pulse wave signal in which the pulse wave has been most favorably measured, and selects this observation signal (Step S203).

Here, the observation signal selected at Step S203 is judged to have been hardly influenced by body-movement noise (acceleration components) because of the user US standing still, and regarded as having a signal waveform equivalent or approximate to that of the observation signal stored in the standing still pulse wave amplitude recording section 65 in the above-described standing still pulse wave measuring operation (refer to FIG. 6).

Next, the signal processing section 70 searches for an extreme value from the selected observation signal for each waveform, and calculates an extreme value interval (Step S204).

Specifically, in a case where the observation signal selected at Step S203 has a signal waveform such as that depicted in FIG. 8, the signal processing section 70 calculates a time indicating a difference value between times Ta and Tb, where the amplitude in each waveform included in the observation signal has a minimum value Pmin, as an extreme value interval.

Note that the extreme value interval calculating operation at Step S204 may be performed for a waveform at an arbitrary time (that is, a typical waveform) among waveforms included in the selected observation signal. Alternatively, an average value obtained by averaging a plurality of extreme value intervals calculated for a plurality of waveforms included in the observation signal in a predetermined period may be used, or a median value extracted from a distribution of a plurality of extreme value intervals may be used.

Next, based on the extreme value interval calculated at Step S204, the signal processing section 70 calculates a pulse rate per unit time (for example, one minute) (Step S205).

Specifically, when the time unit of the extreme value interval calculated from the selected observation signal is seconds, the signal processing section 70 divides 60 by the extreme value interval for conversion to a pulse rate for one minute.

Next, the signal processing section 70 causes the calculated pulse rate to be displayed on the display section 80 by using numerical information, image information, or the like, so that the user US is provided with or notified of the calculated pulse rate (Step S206).

Next, when continuing the pulse rate measurement, the signal processing section 70 returns to Step S201. Conversely, when discontinuing (ending) the pulse rate measurement, the signal processing section 70 ends the movement pulse wave measuring operation (Step S207).

At Step S203, a method has been described in which the signal processing section 70 selects one observation signal having the maximum amplitude among the observation signals of the plurality of pulse waves obtained at the respective observation sites. However, the present invention is not limited thereto.

In the biological information detecting method according to the present invention, for example, an extreme value interval may be calculated for each of the plurality of observation signals and converted to a pulse rate, and an average value, a medium value, or the like may be eventually calculated for these pulse rates and provided to the user US.

At Step S202, when judged that the amplitude of each of the obtained acceleration signals in three axial directions is larger than the threshold value, the signal processing section 70 judges that this observation signal has been influenced by body-movement noise (acceleration components).

In this case, processing for reducing the influence of body-movement noise described below is performed.

Specifically, among the plurality of pulse wave observation signals obtained at Step S201, the signal processing section 70 first regards (Judges) an observation signal having an amplitude most approximate to the amplitude of the observation signal in the standing still state as a pulse wave signal least influenced by body-movement noise, and selects this observation signal (Step S208).

By this observation signal selection processing, a risk that a pulse wave signal is almost eliminated by body-movement noise can be reduced. In other words, a situation can be avoided where a pulse wave signal is erased by body-movement noise and cannot be judged.

Here, the observation signal selected at Step S208 has a signal waveform with pulse wave components and a body-movement noise components mixed together such as that represented by a solid line in FIG. 9A.

In FIG. 9A, a dotted line represents a pulse wave signal not containing body-movement noise or in the state where body-movement noise is substantially negligible (for example, the observation signal obtained by the standing still pulse wave measuring operation; hereinafter referred to as a “reference pulse-wave signal”).

Here, in the case of the observation signal (solid line) depicted in FIG. 9A, due to the influence of body-movement noise, the phase of the observation signal has been shifted from the phase of the reference pulse-wave signal.

Next, the signal processing section 70 performs time lag and rotation angle estimation processing for estimating a time difference (time lag) between the time when the user US start moving and the time when the influence of acceleration due to the movement appears on a composite waveform of the acceleration signal and the pulse wave observation signal selected at Step S208, and also estimating a rotation angle corresponding to an angular difference between the main blood flow direction of the observation site and the axial direction of the acceleration signal (Step S300).

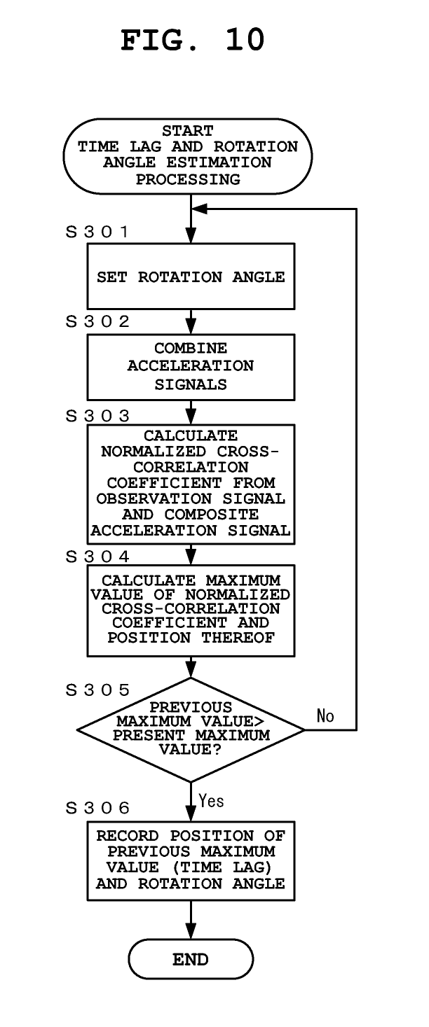

FIG. 10 is a flowchart of the time lag and rotation angle estimation processing that is performed in the movement pulse wave measuring operation according to the present embodiment.

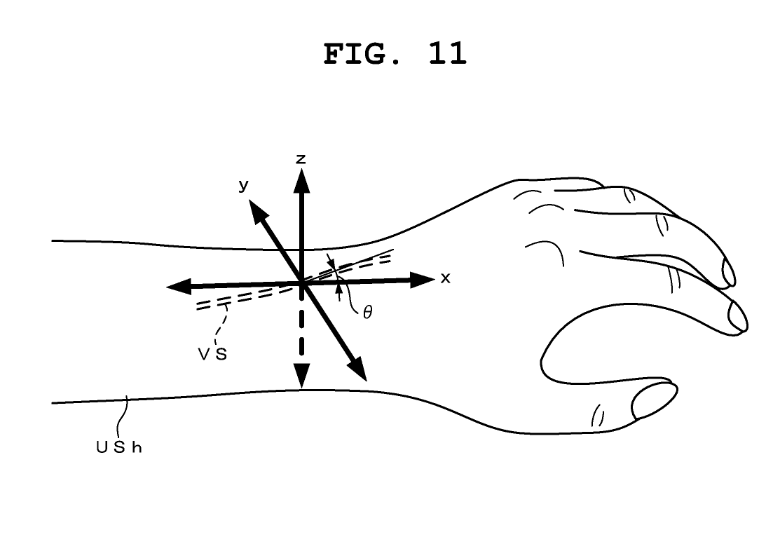

FIG. 11 is a conceptual diagram for describing three axial directions defined in the movement pulse wave measuring operation according to the present embodiment.

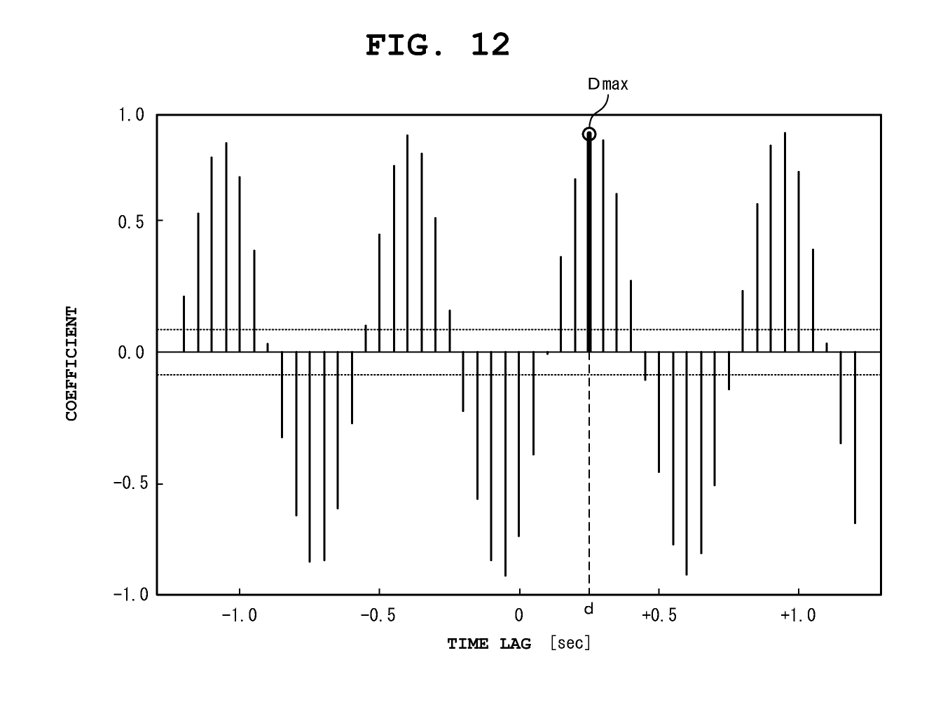

FIG. 12 is a diagram depicting an example of a normalized cross-correlation coefficient calculated by the time lag and rotation angle estimation processing according to the present embodiment.

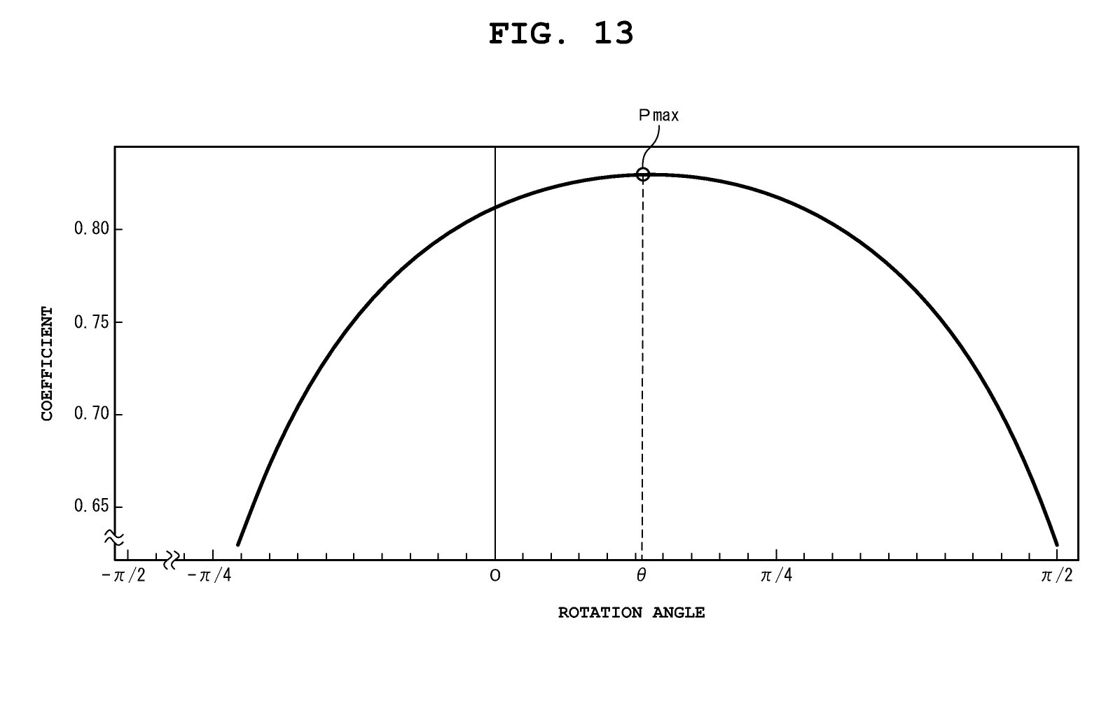

FIG. 13 is a diagram depicting an example of the transition of a rotation angle and a maximum value obtained by the time lag and rotation angle estimation processing according to the present embodiment.

First, the combining of acceleration signals is described.

A composite acceleration signal where the above-described time lag has been taken into consideration can be calculated by using the following Equation (1).

Equation (1)

A(t)=c1×Aₓ(t−d1)+c2×A_{y}(t−d2)+c3×A_{z}(t−d3) (1)

Here, A(t) is a composite acceleration signal obtained by combining acceleration signals in three axial directions, and corresponds to an acceleration component included in an observation signal.

Ax, Ay, and Az are acceleration signals in the x-axis direction, the y-axis direction, and the z-axis direction, respectively, and t represents time.

c1, c2, and c3 are proportionality coefficients on the acceleration signals Ax, Ay, and Az, and are coefficients for setting the amplitude of the composite acceleration signal A(t).

d1, d2, and d3 each represent a time difference (a time lag) by the time when the influence of a relevant one of the acceleration signals Ax, Ay, and Az appears on the pulse wave observation signal. Here, as for the x axis, the y axis, and the z axis, for example, the major axis direction of the wrist USh (the arm extending direction; the lateral direction in the drawing) is defined as x-axis direction, the minor axis direction of the wrist USh (the arm width direction; the direction from upper left to lower right in the drawing) orthogonal to the x-axis direction is defined as y-axis direction, and the front and back direction of the wrist USh (the vertical direction in the drawing) orthogonal to the x-axis direction and the y-axis direction is defined as z-axis direction, as depicted in FIG. 11.

That is, the x axis and the y axis are defined in a direction along the skin surface SF of the wrist USh.

The composite acceleration signal A(t) obtained by combining the acceleration signals in the three axial directions of x, y, and z defined in FIG. 11 can be theoretically calculated by using Equation (1) above.

However, as a result of various verifications, the inventor of the present application has found that (a) the acceleration signal Az in the z axis direction hardly has an influence on a pulse wave signal, (b) as for the time lags d1, d2, and d3, approximately equal values can be obtained hardly depending on the axial direction, and (c) the composite acceleration signal A(t) can be calculated by rotating the acceleration signal Ax in the x-axis direction and the acceleration signal Ay in the y-axis direction, and thereby a value approximately equal to the true (original) composite acceleration value is obtained.

Here, the ratio of coefficient between the acceleration signals in the x-axis direction and the y-axis direction can be defined by axial rotation because the main blood flow direction (that is, the extending direction of a blood vessel VS depicted in FIG. 11) in a plurality of arteries and capillary vessels present under the skin of an observation site (a layer under the skin surface SF) is different for each observation site.

That is, a rotation angle θ depicted in FIG. 11 corresponds to an angular difference between the main blood flow direction of an observation site and the axial direction of an acceleration signal (the x-axis direction in FIG. 11).

Based on this verification, the composite acceleration signal A(t) in three axial directions described in Equation (1) can be calculated by using the following Equation (2).

Equation (2)

A(t)=c×cos θ×Aₓ(t−d)−c×sin θ×A_{y}(t−d) (2)

In the time lag and rotation angle estimation processing at Step S300, with the proportionality coefficient c on the acceleration signals Ax and Ay in Equation (2) fixed as c=1, the value of the time lag d and the value of the rotation angle θ between the acceleration signals Ax and Ay are estimated.

In the time lag and rotation angle estimation processing, the signal processing section 70 first sets a rotation angle θ between acceleration signals in the x-axis direction (x axis) and the y-axis direction (y axis) (Step S301), as depicted in FIG. 10.

This rotation angle θ is sequentially updated (incremented or decremented) by a predetermined angle in a range of −90° (=−π/2) to +90° (=π/2) every time the series of processing (Steps S301 to S305) described below is repeated. As a result, a search for an optimum rotation angle θ is made.

Here, for the purpose of simplifying description, a case is described in which the rotation angle θ is set at 0° as an example of an initial value, and the angle is sequentially incremented at predetermined intervals from 0° to +90°.

The signal processing section 70 sets a state where no time lag has occur (that is, the time lag d=0) as an initial state.

Next, based on the rotation angle θ set as an initial value (=0°) and the proportionality coefficient c=1 and the time lag d, the signal processing section 70 combines the acceleration signal Ax(t) in the x-axis direction and the acceleration signal Ay(t) in the y-axis direction by using Equation (2) (Step S302).

Here, the composite acceleration signal A(t) generated at Step S302 has a signal waveform such as that represented by a solid line in FIG. 9B. In FIG. 9B, a dotted line represents the above-described reference pulse-wave signal.

Next, based on the observation signal selected at Step S208 and the composite acceleration signal A(t) generated at Step S302, the signal processing section 70 calculates a normalized cross-correlation coefficient for the time lag d (Step S303).

The normalized cross-correlation coefficient calculated at Step S303 is represented as depicted in FIG. 12.

Here, there is a causal relation in which acceleration occurred when the user US is moving influences on a pulse wave observation signal after the elapse of some time. This time represents the time lag.

In the normalized cross-correlation coefficient depicted in FIG. 12, the signal processing section 70 sequentially updates the value of the time lag at predetermined intervals in a direction in which the above-mentioned causal relation is established (in the case of FIG. 12, the direction in which the time lag is changed from 0 to positive). As a result, the signal processing section 70 searches for the value of the time lag with which the correlation coefficient becomes a maximum value Dmax for the first time.

The signal processing section 70 then extracts the time lag d when the correlation coefficient becomes the maximum value Dmax for the first time, and stores the time lag d in a predetermined storage area of the memory section 60 (Step S304).

In FIG. 12, the position where the correlation coefficient becomes the maximum value Dmax is represented by a bold line.

Here, in the normalized cross-correlation coefficient depicted in FIG. 12, as for the range in which the time lag d is extracted, for example, the value of the time lag d may be sequentially incremented and the processing may end when the correlation coefficient becomes the maximum value Dmax. Alternatively, with the value of the time lag d defined as not becoming a specific time, for example, one second or more, the signal processing section 70 may calculate the normalized cross-correlation coefficient by that time and then the maximum value Dmax of the correlation coefficient may be found within that time range.

Next, the signal processing section 70 judges whether the present maximum value of the correlation coefficient calculated at Step S304 is smaller than the maximum value previously calculated (Step S305).

Specifically, the signal processing section 70 reads out the present and previous maximum values of the correlation coefficients from the memory section 60. Then, when judged that the present maximum value is smaller than the previous maximum value (that is, the previous maximum value is larger than the present maximum value), the signal processing section 70 stores (records) the time lag d at the position of the previous maximum value and the previous rotation angle θ in a predetermined storage area of the memory section 60 (Step S306), and ends the time lag and rotation angle estimation processing.

At Step S305, when judged that the maximum value of the present correlation coefficient is equal to or larger than the previous maximum value, the signal processing section 70 stores the time lag d at the position of the present maximum value and the present rotation angle θ in a predetermined storage area of the memory section 60.

Then, after returning to Step S301, the signal processing section 70 resets the rotation angle θ, and again performs the series of search processing (Steps S301 to S305) described above.

At Step S305, in the case of initial judgment processing, there is no previous maximum value. Therefore, the signal processing section 70 unconditionally returns to Step S301. Then, after resetting the rotation angle θ, the signal processing section 70 again performs the series of search processing (Steps S301 to S305) described above.

In the above-described time lag and rotation angle estimation processing, the judgment processing at Step S305 is applied because a result has been obtained from verification by the inventor of the present invention that the maximum value of the correlation coefficient has unimodality with respect to the change of the rotation angle θ. However, the present invention is not limited thereto.

For example, a method may be applied in which a normalized cross-correlation coefficient is calculated for all rotation angles θ in the range of −90° to +90° and, from the calculation results, the time lag d at the position where the maximum value of the correlation coefficient becomes maximum (Pmax in the drawing) and the rotation angle θ at that time are selected and stored in the memory section 60.

A relation (transition) between the rotation angle θ and the maximum value of the correlation coefficient when the above-described methodology is applied is established as depicted in FIG. 13.

In the present embodiment, the rotation angle θ is updated in a setting range of 180° from −90° to +90°. However, the present invention is not limited thereto. The setting range of the rotation angle θ is only required to be at least 180° and, for example, 360° (all-around) may be set as the setting range.

Next, based on the time lag d and rotation angle θ estimated in the time lag and rotation angle estimation processing, the signal processing section 70 estimates a proportionality coefficient for setting the amplitude of the composite acceleration signal. Then, the signal processing section 70 performs amplitude estimation processing for generating a signal approximate to the true pulse wave signal from which the influence of body-movement noise due to the movement of the user US has been removed (Step S400).

That is, in the amplitude estimation processing, processing is performed in which the proportionality coefficient c between the acceleration signals Ax and Ay in the x and y directions, respectively, is estimated as a coefficient for setting the amplitude of the composite acceleration signal A(t) in Equation (2).

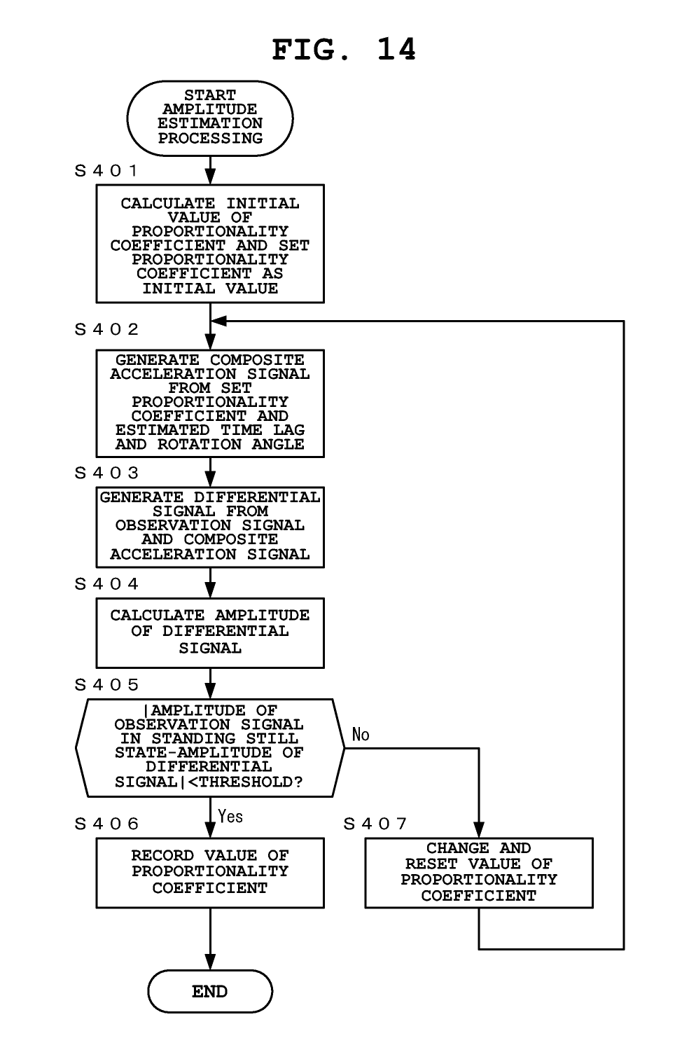

FIG. 14 is a flowchart of the amplitude estimation processing which is performed in the movement pulse wave measuring operation according to the present embodiment.

In the amplitude estimating processing, as depicted in FIG. 14, the signal processing section 70 first sets the proportionality coefficient c between the acceleration signals Ax and Ay in the x and y directions in Equation (2) (Step S401).

Here, first, in Equation (2), by applying the values of the time lag d and the rotation angle θ of the acceleration signal estimated in the time lag and rotation angle estimation processing, the signal processing section 70 calculates the amplitude of the composite acceleration signal A(t) when the proportionality coefficient c is set at 1.

Then, the signal processing section 70 compares the amplitude of the composite acceleration signal A(t) and the amplitude of the pulse wave observation signal selected at Step S208. Then, the signal processing section 70 calculates the value of the proportionality coefficient c with which the amplitude of the composite acceleration signal A(t) is equal to the amplitude of the observation signal as an initial value of the proportionality coefficient c, and sets the proportionality coefficient c at this initial value.

Next, based on the set proportionality coefficient c and the values of the time lag d and the rotation angle θ of the acceleration signal estimated in the above-described time lag and rotation angle estimation processing, the signal processing section 70 generates a composite acceleration signal A(t) by using Equation (2) (Step S402). Next, the signal processing section 70 takes a difference value between the pulse wave observation signal selected at Step S208 and the composite acceleration signal A(t) generated at Step S402 to generate a signal indicating the difference value as a differential signal (Step S403).

Next, the signal processing section 70 calculates the amplitude of the generated differential signal (Step S404).

Next, the signal processing section 70 judges whether an absolute value of a difference value between the amplitude of the pulse wave observation signal in the standing still state obtained in the standing still pulse wave measuring operation and the amplitude of the differential signal is smaller than a predetermined threshold value (Step S405).

When judged that the absolute value is smaller than the threshold value, the signal processing section 70 stores (records) the value of the proportionality coefficient c in a predetermined storage area of the memory section 60 (Step S406), and ends the amplitude estimation processing.

Conversely, when judged that the absolute value is equal to or larger than the threshold value at Step S405, the signal processing section 70 resets and updates the value of the proportionality coefficient c to a different value (Step S407).

Then, after returning to Step S402, the signal processing section 70 again performs the series of processing (Steps S402 to S405) described above.

Here, the value of the proportionality coefficient c to be reset is sequentially increased or decreased at predetermined intervals.

Here, the differential signal generated in the processing for generating a differential signal (Step S403) has a signal waveform such as that represented by a solid line in FIG. 9C. In FIG. 9C, a dotted line represents the above-described reference pulse-wave signal.

FIG. 9C depicts a case in which, by the series of processing including the time lag and rotation angle estimation processing and the amplitude estimation processing described above being performed, a signal waveform substantially coinciding with the reference pulse-wave signal is obtained as the above-described differential signal.

In the above-described amplitude estimation processing, the method is applied in which whether to repeat the series of processing is judged with reference to the amplitude of the observation signal in the standing still state. However, the present invention is not limited thereto.

For example, a method may be applied in which, when the amplitude of body-movement noise is sufficiently larger than the amplitude of the pulse wave signal, the value of the proportionality coefficient c is sequentially updated to search for the minimum value of the amplitude of the differential signal and, based on the minimum value, whether to repeat the series of processing is judged.

Next, after the time lag and rotation angle estimation processing (Step S300) and the amplitude estimation processing (Step S400) end, the signal processing section 70 regards the generated differential signal as a pulse wave signal, and calculates an extreme value interval, as depicted in FIG. 7 (Step S209).

The extreme value interval calculating operation at Step S209 may be performed for a waveform at an arbitrary time (that is, a typical waveform) among waveforms included in the generated differential signal, as in the case of Step S204. Alternatively, an average value obtained by averaging a plurality of extreme value intervals calculated for a plurality of waveforms may be used, or a median value extracted from a distribution of a plurality of extreme value intervals may be used.

Next, based on the extreme value interval calculated at Step S209, the signal processing section 70 calculates a pulse rate for one minute (Step S205).

Then, the signal processing section 70 displays the calculated pulse rate on the display section 80, so that the user US is provided with or notified of the calculated pulse rate (Step S206).

As described above, in the method of the present embodiment where a pulse rate is calculated by body-movement noise components due to an exercise being removed from a pulse wave observation signal obtained while the user US is exercising by photoplethysmography, when the amplitude of acceleration signals in three axial directions obtained at the time of the exercise exceeds a predetermined threshold value, a pulse rate is calculated by using a signal (differential signal) from which acceleration components in a specific direction (the rotation angle θ in an x-y plane including the x axis and the y axis) along the body surface has been removed from the pulse wave observation signal.

Here, in the present embodiment, in order to find the acceleration components to be removed from the pulse wave observation signal, a method is applied in which three parameters including a time difference (time lag d) from the pulse wave observation signal, a coefficient (proportionality coefficient c) for defining the magnitude of the amplitude, and the rotation angle θ of acceleration in each direction are estimated.

Note that the value of each parameter changes according to the movement of the user US, or in other words, the movement status of each site of the body. For example, the influence of the acceleration on the pulse wave observation signal varies depending on the user's state, such as a slow walking state, a fast running state, a state of not swinging arms, and a state of fully swinging arms, and the value of each parameter changes. Accordingly, the value of each parameter is required to be estimated every time pulse wave measurement is performed.

The present embodiment has a structure in which a plurality of light-emitting elements and light-receiving elements are arranged in a measurement area, and multipoint observation is performed to measure pulse waves at a plurality of different observation sites in the measurement area. When the amplitudes of acceleration signals in three axial directions obtained at the time of exercise exceed a predetermined threshold value, a pulse wave observation signal of an observation site with less influence of body-movement noise is reselected to calculate a pulse rate.

In this selection of an observation signal, a method is adopted in which, based on the amplitude of an observation signal obtained at each observation site in the standing still state, an observation signal least influenced by body-movement noise is selected.

As such, in the present embodiment, by an acceleration signal (acceleration components) calculated based on newly estimated parameters being removed from a pulse wave observation signal during exercise, a signal (differential signal) having the same phase as that of the true (original) pulse-wave signal can be obtained. Also, based on this differential signal, an instantaneous pulse at the time of exercise can be relatively accurately measured.

### Second Embodiment

Next, a second embodiment of the biological information detecting device according to the present invention is described.

Here, sections and operations equivalent to those of the first embodiment are described with reference to the above-described drawings as appropriate.

As depicted in FIG. 2, the first embodiment has a structure where at least the number of light-emitting elements or light-receiving elements arranged in the measurement area MS of the biological information detecting device 100 is not one, in which an optimum observation signal is selected from a plurality of pulse wave observation signals obtained by multipoint observation.

However, the second embodiment has a structure where one light-emitting element and one light-receiving element are arranged in the measurement area MS, in which a method is used where only one pulse wave observation signal is obtained from one observation site (single-point observation).

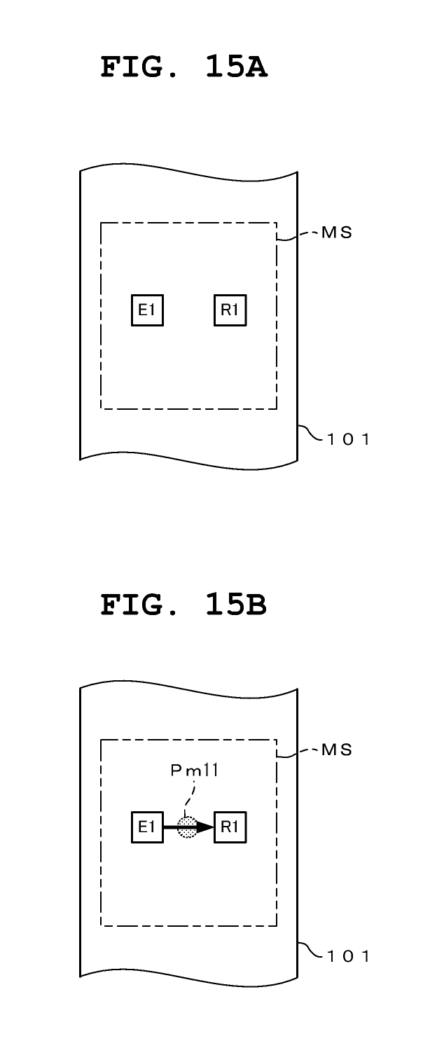

FIG. 15A and FIG. 15B are schematic diagrams depicting a structural example of the measurement surface of a biological information detecting device according to the second embodiment. FIG. 15A is a schematic diagram depicting an example of the arrangement of light-emitting elements and light-receiving elements, and FIG. 15B is a conceptual diagram depicting a pulse wave observation site.

The biological information detecting device according to the second embodiment has the same structure as the first embodiment (refer to FIG. 1) except that one light-emitting element E1 and one light-receiving element R1 are arranged in the measurement area MS of the device bode 101, as depicted in FIG. 15A.

That is, in the present embodiment, light-emitting element and light-receiving element are arranged such that they have a one-to-one relation.

In the structure of the biological information detecting device depicted in FIG. 3, the light-emission control section 15 controls the light-emitting element E1 to emit light with a predetermined light-emission intensity to irradiate the observation site Pm11 on the skin surface SF with light as depicted in FIG. 15B, and the light dispersed by blood in blood vessels near the skin surface SF is received by the light-receiving element R1 as reflected light. As a result, a pulse wave observation signal of the observation site Pm11 on the skin surface SF is obtained.

Next, a biological information detecting method according to the present embodiment is described.

Here, operations and processing equivalent to those of the first embodiment are described with reference to the above-described drawings as appropriate.

In the biological information detecting method according to the present embodiment, a standing still pulse wave measuring operation and a movement pulse wave measuring operation are preformed, as with the first embodiment.

First, in the standing still pulse wave measuring operation according to the present embodiment, the signal processing section 70 prompts the user US to stand still in the flowchart of FIG. 4 in the first embodiment. Then, the signal processing section 70 causes the light-emitting element E1 to emit light, and its reflected light is received by the light-receiving element R1. As a result, the signal processing section 70 obtains a pulse wave observation signal and an acceleration signal in the standing still state for a predetermined period (Step S101).

Here, since the present embodiment has a structure in which the light-emitting element and the light-receiving element are arranged in a one-to-one relation, only one pulse wave observation signal is obtained from one observation site Pm11 at Step S101 (single-point observation).

Then, when judged at Step S102 that the amplitude of the acceleration signal measured at the time of obtaining the observation signal is equal to or smaller than a predetermined threshold value, the signal processing section 70 judges that the user US is standing still or at rest, and stores the average value of the amplitude of the pulse wave observation signal obtained at Step S101 in the standing still pulse wave amplitude recording section 65 as the amplitude of the observation signal in the standing still state (Step S103).

At Step S102, when judged that the amplitude of the acceleration signal measured at the time of obtaining the observation signal is equal to or larger than the predetermine threshold, the signal processing section 70 judges that the user US is not standing still or at rest, and after performing the operations at Steps S104 and S105, performs the series of processing (Steps S101 to S105) again, as with the first embodiment.

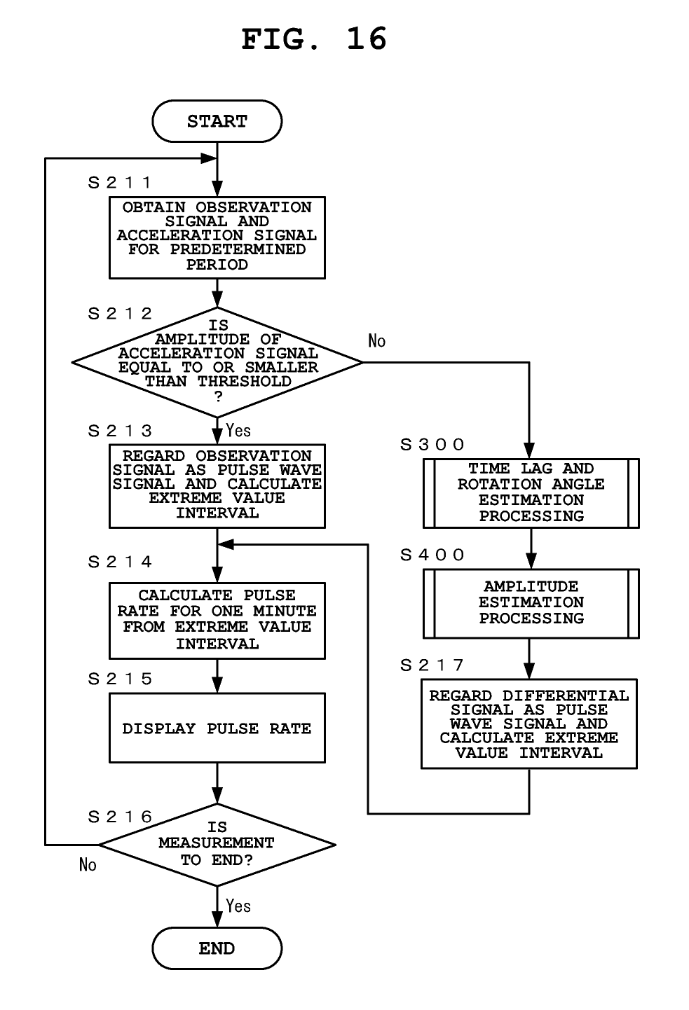

FIG. 16 is a flowchart of a movement pulse wave measuring operation that is performed in the biological information detecting method according to the present embodiment.

In the movement pulse wave measuring operation according to the present embodiment, the observation signal selection processing at Steps S203 and S208 are omitted from the flowchart of FIG. 7 in the first embodiment.

That is, in the movement pulse wave measuring operation according to the present embodiment, the signal processing section 70 obtains a pulse wave observation signal and an acceleration signal when the user US is exercising, for a predetermined period, as depicted in the flowchart of FIG. 16 (Step S211). At this Step S211 as well, only one pulse wave observation signal is obtained by single-point observation.

Then, when judged at Step S212 that the amplitude of the acceleration signal measured when the observation signal is obtained is equal to or smaller than a predetermined threshold value, the signal processing section 70 regards the pulse wave observation signal obtained at Step S211 as a pulse wave signal whose pulse wave has been favorably measured.

Then, the signal processing section 70 calculates an extreme value interval of this observation signal (Step S213).

Next, based on the extreme value interval calculated at Step S213, the signal processing section 70 calculates a pulse rate for one minute (Step S214).

Then, the signal processing section 70 displays the calculated pulse rate on the display section 80, so that the user US is provided with or notified of the calculated pulse rate (Step S215).

Conversely, when judged at Step S212 that the amplitude of the obtained acceleration signal is larger than the threshold value, the signal processing section 70 performs processing similar to that of the first embodiment. That is, the signal processing section 70 performs the time lag and rotation angle estimation processing (Step S300) for estimating a time lag by the time when the influence of the acceleration appears on the composite acceleration signal A(t) and the pulse wave observation signal, and a rotation angle corresponding to an angular difference between the main blood flow direction of the observation site and the axial direction of the acceleration signal, and also performs the amplitude estimation processing (Step S400) for generating a signal approximate to the true pulse-wave signal from which the influence of body-movement noise has been removed, as a differential signal.

Then, the signal processing section 70 regards the differential signal generated by the series of processing at Steps S300 and S400 as a pulse wave signal, and calculates an extreme value interval (Step S217).

Next, based on the extreme value interval calculated at Step S217, the signal processing section 70 calculates a pulse rate for one minute (Step S214).

Next, the signal processing section 70 displays the calculated pulse rate on the display section 80, so that the user US is provided with or notified of the calculated pulse rate (Step S215).

Then, when continuing the pulse rate measurement, the signal processing section 70 again performs the series of processing (Steps S211 to S217).

As such, in the present embodiment as well, from a pulse wave observation signal during exercise, an acceleration signal (acceleration components) is removed which has been calculated based on newly estimated three parameters, that is, a time difference from the pulse wave observation signal (time lag d), a coefficient for defining the magnitude of amplitude (proportionality coefficient c), and the rotation angle θ of an acceleration signal in each axial direction, whereby a signal (differential signal) having the same phase as that of the true (original) pulse-wave signal can be obtained, as with the first embodiment described above. Also, based on this differential signal, an instantaneous pulse at the time of exercise can be relatively accurately measured.

Here, the present embodiment has a structure in which one light-emitting element and one light-receiving element have been arranged in a measurement area, in which single-point observation for calculating a pulse wave of one observation site is applied. Therefore, an instantaneous pulse at the time of exercise can be measured by simple processing based on a single pulse wave observation signal.

In each of the above-described embodiments, the biological information detecting device 100 has a wristwatch-type shape, and the device body 101 including the measurement area MS is worn such that it comes close contact with the back side of the wrist USh of the user US. However, the present invention is not limited thereto. For example, the device may be worn such that it comes close contact with the palm side.

As explained in the descriptions of the embodiments, when worn on the back side of the wrist, the device is resistant to the influence of change in the wearing status (the contacting status of the measurement area with respect to the skin surface) due to the rise of a tendon of the wrist or the like, compared with the case where the device is worn on the palm side. Thus, an observation signal can be favorably obtained.

Also, in each of the above-described embodiments, the biological information detecting device 100 has a wristwatch-type shape, and is worn on the wrist USh of the user US. However, the present invention is not limited thereto.

That is, in the present invention, it is only required that a biological information detecting device in which a light-emitting element and a light-receiving element have been arranged in a predetermined pattern in the measurement area MS is worn such that it comes close contact with a site where a pulse wave when the human body is moving can be observed.

For example, the device may have a shape by which it can be worn or held with a belt or the like on the observation site such as the above-described wrist, an arm part such as brachial, a finger part except a finger tip, an earlobe, or an ankle.

While the present invention has been described with reference to the preferred embodiments, it is intended that the invention be not limited by any of the details of the description therein but includes all the embodiments which fall within the scope of the appended claims.

What is claimed is:

1. A biological information detecting device comprising:
a detecting section which outputs an observation signal detected based on a pulse wave of at least one observation site of a user;
an acceleration measuring section which outputs a plurality of acceleration signals which are corresponding to a plurality of different axial directions and are measured along with a movement of the user;
a parameter estimating section which estimates, based on a comparison between the observation signal and a composite acceleration signal obtained by combining the plurality of acceleration signals based on a plurality of parameters, a specific value of each of the parameters corresponding to acceleration components obtained in response to the movement of the user; and
a pulse rate calculating section which calculates a pulse rate of the user as a biological information from a difference value obtained by subtracting a specific composite acceleration signal corresponding to the specific value of each of the parameters from the observation signal.

2. The biological information detecting device according to claim 1, wherein the parameter estimating section estimates the specific value of each of the parameters based on values of cross-correlation coefficients between a plurality of composite acceleration signals different from each other and the observation signal when the parameters are set at a plurality of different values.

3. The biological information detecting device according to claim 2, wherein the parameter estimating section obtains a value of a cross-correlation coefficient for each of the plurality of composite acceleration signals, and estimates a value of each of the parameters when the value of the cross-correlation coefficient is maximum as the specific value of each of the parameters.

4. The biological information detecting device according to claim 1, wherein the plurality of parameters include a first parameter corresponding to a time difference between a time when the user starts moving and a time when an influence of movement occurs to the observation signal, and a second parameter corresponding to an angular difference between each of the axial directions of the plurality of acceleration signals and a main blood flow direction at the observation site, and
wherein the parameter estimating section estimates, as the specific value, a specific value of the first parameter and a specific value of the second parameter.

5. The biological information detecting device according to claim 4, further comprising:
a storage section which stores, as amplitude in a standing still state, a value of amplitude of the observation signal detected by the detecting section in the standing still state where the user is not moving,
wherein the plurality of parameters further include a third parameter as a proportionality coefficient for setting amplitude of the composite acceleration signal, and
wherein the parameter estimating section estimates, as the specific value, a specific value of the third parameter based on a comparison between the amplitude in the standing still state and amplitude of a signal indicating the difference value between the observation signal and the composite acceleration signal.

6. The biological information detecting device according to claim 5, wherein the plurality of different axial directions have x axis and y axis in directions orthogonal to each other along a body surface of the observation site of the user and z axis in a direction orthogonal to the x axis and the y axis; and the acceleration measuring section obtains a first acceleration signal in a direction of the x axis, a second acceleration signal in a direction of the y axis, and a third acceleration signal in a direction of the z axis as the plurality of acceleration signals, and
wherein the parameter estimating section estimates the specific value of each of the parameters by using at least the first acceleration signal and the second acceleration signal.

7. The biological information detecting device according to claim 5, wherein the detecting section outputs a plurality of observation signals based on each pulse wave of a plurality of observation sites of the user which are different from each other,
wherein the biological information detecting device further comprises an observation signal selecting section which selects a specific observation signal having amplitude most approximate to the amplitude in the standing still state from among the plurality of observation signals, and
wherein the parameter estimating section estimates specific values of the plurality of parameters based on the specific observation signal.

8. The biological information detecting device according to claim 7, wherein the observation signal selecting section compares amplitude of each of the plurality of acceleration signals with a predetermined threshold value, and selects the specific observation signal from the plurality of observation signals, when the amplitude of each of the plurality of acceleration signals is larger than the threshold value.

9. The biological information detecting device according to claim 7, wherein the detecting section includes a light-emitting section which irradiates each of the plurality of observation sites with light, and a light-receiving section which receives light irradiated from the light-emitting section and reflected from each of the plurality of observation sites and outputs the plurality of observation signals,
wherein the light-emitting section includes one or plurality of light-emitting elements which emit light,
wherein the light-receiving section includes one or plurality of light-receiving elements which receive light, and
wherein the light-emitting section and the light-receiving section include at least the plurality of light-emitting elements or the plurality of light-receiving elements.

10. The biological information detecting device according to claim 1, further comprising:
a display section which displays the pulse rate calculated by the pulse rate calculating section.

11. A biological information detecting method comprising:
a step of obtaining an observation signal based on a pulse wave of at least one observation site of a user, and obtaining a plurality of acceleration signals which are corresponding to a plurality of different axial directions along with a movement of the user;
a step of estimating, based on a comparison between the observation signal and a composite acceleration signal obtained by combining the plurality of acceleration signals based on a plurality of parameters, a specific value of each of the parameters corresponding to acceleration components obtained in response to the movement of the user included in the observation signal; and
a step of calculating a pulse rate of the user as a biological information from a difference value obtained by subtracting a specific composite acceleration signal corresponding to the specific value of each of the parameters from the observation signal.

12. The biological information detecting method according to claim 11, wherein the step of estimating the specific value of each of the parameters is performed based on values of cross-correlation coefficients between a plurality of composite acceleration signals different from each other and the observation signal when the parameters are set at a plurality of different values.

13. The biological information detecting method according to claim 12, wherein the step of estimating the specific value of each of the parameters is performed such that a value of a cross-correlation coefficient is obtained for each of the plurality of composite acceleration signals, and a value of each of the parameters when the value of the cross-correlation coefficient is maximum is estimated as the specific value of each of the parameters.

14. The biological information detecting method according to claim 11, wherein the plurality of parameters include a first parameter corresponding to a time difference between a time when the user starts moving and a time when an influence of movement occurs to the observation signal, and a second parameter corresponding to an angular difference between each of the axial directions of the acceleration signals and a main blood flow direction at the observation site, and
wherein a specific value of the first parameter and a specific value of the second parameter are estimated as specific values of the plurality of parameters.

15. The biological information detecting method according to claim 14, wherein the plurality of parameters further include a third parameter as a proportionality coefficient for setting amplitude of the composite acceleration signal,
wherein a value of amplitude of the observation signal detected in a standing still state where the user is not moving is stored as amplitude in the standing still state, and
wherein a specific value of the third parameter is estimated as a specific value of the plurality of parameters based on a comparison between the amplitude in the standing still state and amplitude of a signal indicating the difference value between the observation signal and the composite acceleration signal.

16. A non-transitory computer-readable storage medium having stored thereon a biological information detection program that is executable by a computer, the program being executable by the computer to perform functions comprising:
processing for obtaining an observation signal based on a pulse wave of at least one observation site of a user, and obtaining a plurality of acceleration signals which are corresponding to a plurality of different axial directions set in advance along with a movement of the user;
processing for estimating, based on a comparison between the observation signal and a composite acceleration signal obtained by combining the plurality of acceleration signals based on a plurality of parameters, a specific value of each of the parameters corresponding to acceleration components obtained in response to the movement of the user included in the observation signal; and
processing for calculating a pulse rate of the user as a biological information from a difference value obtained by subtracting a specific composite acceleration signal corresponding to the specific value of each of the parameters from the observation signal.

17. The non-transitory computer-readable storage medium according to claim 16, wherein the processing for estimating the specific value of each of the parameters is performed based on values of cross-correlation coefficients between a plurality of composite acceleration signals different from each other and the observation signal when the parameters are set at a plurality of different values.

18. The non-transitory computer-readable storage medium according to claim 16, wherein the plurality of parameters include a first parameter corresponding to a time difference between a time when the user starts moving and a time when an influence of movement occurs to the observation signal, and a second parameter corresponding to an angular difference between each of the axial directions of the acceleration signals and a main blood flow direction at the observation site, and
wherein a specific value of the first parameter and a specific value of the second parameter are estimated as specific values of the plurality of parameters.

19. The non-transitory computer-readable storage medium according to claim 18, wherein the plurality of parameters further include a third parameter as a proportionality coefficient for setting amplitude of the composite acceleration signal,
wherein a value of amplitude of the observation signal detected in a standing still state where the user is not moving is stored as amplitude in the standing still state, and
wherein a specific value of the third parameter is estimated as a specific value of the plurality of parameters, based on a comparison between the amplitude in the standing still state and amplitude of a signal indicating the difference value between the observation signal and the composite acceleration signal.
